(19) United States
(12) Patent Application Publication
(10) Pub. No.: US 2005/0288585 A1
(43) Pub. Date: Dec. 29, 2005

(54) Flexible physiological cycle detection and use for ultrasound

(75) Inventors: Zafer Zamboglu, Mountain View, CA (US); Ismayil Guracar, Redwood City, CA (US); John F. Kubel, Sunnyvale, CA (US)

Correspondence Address: Siemens Corporation;Attn: Elsa Keller, Legal Administrator, Intellectual Property Department, 170 Wood Avenue South, Iselin, NJ, 08830, US

(73) Assignee: Siemens Medical Solutions USA, Inc.

(21) Appl. No.: 10/876,189

(22) Filed: Jun. 24, 2004

(51) Int. Cl. A61B 8/00

(52) U.S. Cl. 600/438

(57) ABSTRACT

Processing is optimized for specific sources or conditions for physiological waveforms. For example, parameters are optimized for processing different human and non-human physiological waveforms. Filter cutoff frequencies, amplitude thresholds, delay time periods and/or other parameters for trigger detection and/or waveform generation are optimized. The optimization may be performed using a single user input, such as switching between different physiological cycle processes in response to one-touch by a user. For example, the system switches between a human ECG trigger detection process and associated parameters and a rodent ECG trigger detection process and associated parameters with the touch of a button. Switching between different types of human physiological cycle processes, such as processes for normal and abnormal functioning, is alternatively or additionally provided. More inclusive settings may be used for automatically identifying a characteristic of a physiological cycle waveform. Once the characteristic is identified, such as a characteristic identifying a human waveform associated with a particular abnormal function or a non-human waveform, the processing associated with the physiological cycle signals is optimized for use with the detected type of source or condition.

### BACKGROUND

The present invention relates to the use of physiological cycle information in medical imaging. In particular, flexible use of cycle detection or processing in ultrasound imaging is provided.

Ultrasound imaging systems have inputs for receiving electrocardiogram (ECG) or breathing cycle signals. An ECG or breathing cycle monitor or sensor provides the input signals. Alternatively, ultrasound data, such as Doppler flow information, is used to obtain signals representing a physiological cycle. The ultrasound imaging system then processes the signals, such as filtering the signals. The processed information is used for triggering, such as triggering contrast agent imaging every occurrence of an R-wave of an ECG cycle. Alternatively or additionally, the ultrasound imaging system processes the signals to generate a waveform representing the cycle on a display. However, the physiological cycles of different types of patients may have different characteristics. Processing, such as filter cutoff frequencies and amplitude thresholds, may be more accurate for one type of patient or patient condition than another. For example, ECG processing may be optimized for human physiology of a normal ECG waveform. Where the sensor is applied to a non-human, such as a mouse, the ECG processing may be less optimized and fail to operate properly. Rodent ECG waveforms may have different characteristics due to anatomical and physiological differences. As a result, R-wave detection or other processes may fail.

### BRIEF SUMMARY

By way of introduction, the preferred embodiments described below include methods and systems for processing signals representing a physiological cycle with an ultrasound system. The processing is optimized for specific sources or conditions. For example, parameters are optimized for processing different human and non-human physiological waveforms. Filter cutoff frequencies, amplitude thresholds, delay time periods and/or other parameters for trigger detection and/or waveform generation are optimized. In one embodiment, the optimization is performed using a single user input, such as switching between different physiological cycle processes in response to one-touch or activation by a user. For example, the system switches between a human ECG trigger detection process and associated parameters and a rodent ECG trigger detection algorithm and associated parameters with the touch of a button. Switching between different types of human physiological cycle processes, such as processes for normal and abnormal functioning, is alternatively or additionally provided.

In other embodiments, more inclusive settings are used for identifying a characteristic of a physiological cycle waveform. Once the characteristic is identified, such as a characteristic identifying a human waveform associated with a particular abnormal function or a non-human waveform, the processing associated with the physiological cycle signals is optimized for use with the detected type of source or condition.

In a first aspect, a method is provided for processing signals representing a physiological cycle with an ultrasound system. Signals representative of at least a portion of a physiological cycle are received. Two or more different parameters associated with respective two or more different physiological cycle characteristics are provided. One of the different parameters is selected. The physiological cycle signals are processed as a function of the selected parameter.

In a second aspect, a system is provided for optimizing an ultrasound system for a specific physiological waveform. An input is operable to receive signals representing at least a portion of a physiological cycle. A memory is operable to store different physiological waveform processes for different physiological waveforms of a same type. A processor is operable to implement at least one of the different physiological waveform processes on the signals.

In a third aspect, a method is provided for processing signals representing a physiological cycle with an ultrasound system. Signals representative of at least a portion of a physiological cycle are received. A characteristic of the signals is determined. The signals are processed. The processing of the signals is set as a function of the characteristic.

In a fourth aspect, a method is provided for processing signals representing a physiological cycle with an ultrasound system. Signals representative of at least a portion of a physiological cycle are received. Different parameters associated with different physiological cycle characteristics are provided. The processing of the physiological signals is switched between the different parameters in response to user input.

The present invention is defined by the following claims, and nothing in this section should be taken as a limitation on those claims. Further aspects and advantages of the invention are discussed below in conjunction with the preferred embodiments and may be later claimed in combination or independently. Various features and advantages discussed herein may or may not be provided by different embodiments of the invention.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system with a physio-system for optimizing a physiological waveform process;

FIG. 2 is a flow chart diagram of one embodiment of a method for processing signals representing a physiological cycle with an imaging system;

FIG. 3 is a graphical representation of one embodiment of a single cycle of a human ECG waveform;

FIG. 4 is a graphical representation of one embodiment of a single cycle of a rodent ECG waveform; and

FIG. 5 is a flow chart diagram of one embodiment of a method for using a hold-off time period and triggering based on a physiological signal.

### DETAILED DESCRIPTION OF THE DRAWINGS AND PRESENTLY PREFERRED EMBODIMENTS

Characteristics of non-standard, such as abnormal or non-human physiological signals are compensated for in a physiological module of an imaging system. For example, in human ECG, arrhythmia, tachycardia and/or fetal heart waveforms may be considered abnormal as compared to an adult human typical waveform. As another example, mice, rodents or other non-human animals may have differing physiological signals. A standardized physio-module may have difficulty dealing with the range of possible physiological signals. For example, a physio-triggering process that operates over the full range of human and rodent heart ranges may not work as well as processes optimized for more specific ranges. Typical ECG processes include thresholds for rejecting heart rates that fall outside of a normal range. By increasing the range to operate with the faster mouse heart, the process is more susceptible to erroneous heart rates when processing signals from a human. R-wave sensitive triggering may become erratic. If the triggering algorithm is sensitive to both mouse and human hearts, false triggering for processing human signals may result. By providing different parameters for use in the physiological signal processes, processes are more optimized to specific physiological cycle source or conditions. Optimized processing may make triggering, detection or waveform generation more likely accurate.

In one embodiment, more inclusive processing (e.g., settings more likely to detect characteristics of different sources or conditions) is used for detecting a characteristic of the physiological signals. The characteristic is then used to select a more specific processing. Alternatively or additionally, the system is configured to allow a user to switch between two or more different processes, such as providing one-touch optimization for switching between a normal adult human ECG trigger detection process and a small animal ECG trigger detection process. The processing of physiological signals of different types of animals, such as humans and non-humans, as well as different types of abnormalities, may be improved.

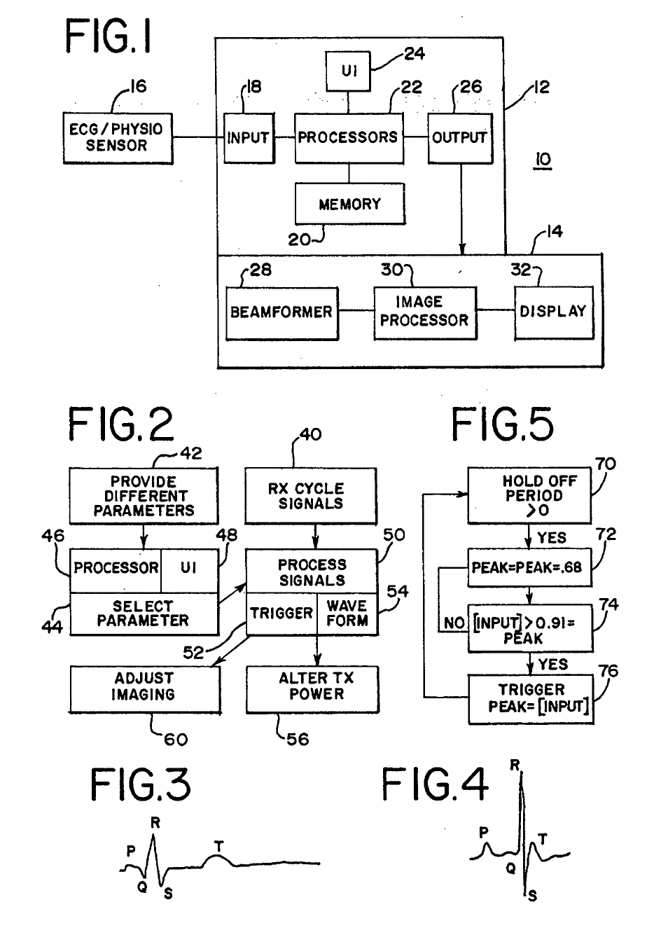

FIG. 1 shows one embodiment of a system 10 for optimizing processing for specific physiological waveforms. The system 10 includes a physio-module 12, an imaging system 14 and an ECG or physiological cycle sensor 16. Additional, different or fewer components may be provided, such as providing the physio-module 12 as separate from or without the imaging system 14. As another example, the sensor 16 may be integrated within the physio-module 12 or signals from a sensor acquired in the past or remotely are transmitted through wireless or wired communications to the physio-module 12. As yet another example, the imaging system 14 senses physiological cycle information and provides the information of the physio-module 12 without a separate sensor 16. In one embodiment, the system 10 is a cart-based, handheld, portable, permanent, or other ultrasound system operable to scan a patient with acoustic energy. In alternative embodiments, the system 10 is an imaging system of different modality, such as MRI, PET or CT.

The sensor 16 is an electrode, set of electrodes, processor, analog-to-digital converter, filters, nasal thermistor, amplifier, microphone, combinations thereof or other now known or later developed sensor for sensing a physiological cycle. For example, the sensor 16 is an ECG sensor for outputting analog or digital signals representing information from a combination or individual ones of electrodes. As another example, the sensor 16 is a respiratory sensor, such as using the transthoracic impedance or temperature within a nasal passage for generating signals representing the breathing cycle. The sensor 16 outputs signals representing at least a portion of a physiological cycle, such as signals representing a cycle at a given time. The signals may include a portion, an entire or multiple cycles over a time period.

The physio-module 12 includes an input 18 for receiving the signals from the sensor 16. Physio-module also includes a memory 20, a processor 22, a user interface 24 and an output 26. Additional, different or fewer components may be provided, such as providing for different processors 22 for implementing different processes on the physiological sensors. For example, different processors 22 are operable to process physiological cycle signals in response to different parameters. As another example, different processors 22 are operable to perform different types of processes based on the same signals, such as one processor 22 for detecting particular events associated with a cycle and another processor 22 for generating a waveform representing the cycle.

The input 18 is a physical connector for connecting with the sensor 16 and/or electronics for providing communications with the sensor 16. Alternatively, the input 18 is a bus or other structure for receiving signals communicated from the imaging system 14 representing a physiological cycle. In one embodiment, the input 18 is a digital input, but analog inputs may be provided. The input 18 is operable to receive signals representative of at least a portion of a physiological cycle. For example, ECG input signals are received.

The memory 20 is a single memory or a plurality of different memories. Any of various now known or later developed memories may be used, such as RAM, removable media, compact disk, magnetic tape, diskette, ROM, cache, buffer or hard drive. In one embodiment, the memory 20 is integrated with each of the processors 22 on a same board or a same chip. In other embodiments, the memory 20 is common to a plurality of processors 22. While the memory 20 is shown within the physio-module 12, the memory 20 may be positioned in-part or entirely elsewhere within the system 10 or remote from the system 10. The memory 20 may store information from various sources, such as from a user interface controller. The memory 20 may also store outbound messages for use by other processors, such as a message carrying heart rate information.

The memory 20 is operable to store data for different physiological waveform processes of respective different physiological waveform characteristics of a same type (e.g. ECG). For example, different processes are stored for ECG waveforms with different characteristics. Different processes may be stored for different breathing waveforms. Different physiological waveform processes may include the same algorithm using different settings, different algorithms, different processing structures, or combinations thereof. Different settings for the different processes may include filter parameters, combinations of filters, heart rate limits, filter bandwidths, cutoff frequencies, trigger hold-off times, trigger thresholds, thresholds, amplitude thresholds or combinations thereof. For example, two different cutoff frequencies for a same type of filter are provided for two different physiological waveform conditions or sources. Different physiological waveforms include normal adult physiological cycles, abnormal adult physiological cycles, normal child physiological cycles, abnormal child physiological cycles, other human physiological cycles, non-human physiological cycles or other groupings. In one embodiment, only two different settings or processes are provided, such as human versus non-human. Greater resolution for more sensitive optimization may provide for three or more different settings or associated different processes. For example, the memory 20 stores settings associated with adult human normal ECG cycles, one or more different adult human abnormal physiological cycles, one or more child or fetus human physiological cycles and/or one or more non-human physiological cycles.

The parameters for processing of physiological signals are optimized for the different kinds of waveforms. The optimum parameters for specific waveforms are loaded into the memory 20 and then used by the processor 22. The received physiological cycle signals are then processed in an optimal way based on the selection of the associated parameters. For example, the R-waves in a rodent ECG may be detected using parameters optimized for rodents so that ultrasound images may be acquired at every heartbeat. R-waves associated with a human ECG are detected using parameters optimized for human heart rate. The optimization allows for the threshold limitation of R-wave frequency in humans without reducing the detectability of R-waves in a rodent.

FIG. 3 shows one cycle of a human ECG, and FIG. 4 shows one cycle of a mouse ECG. The maximum human heartbeat is at about 200 beats per minute, but the mouse heartbeat ranges from 300 to 800 beats per minute. In addition to differences between the cycle intervals, ventricular re-polarization occurs much sooner in a mouse's heart, causing a relatively shorter S-T interval than in a human heart. The atrial and ventricular depolarizations can be further apart in a mouse's heart with respect to the other intervals, resulting in a relatively longer P-R interval. Relatively longer P-R interval and the more similar frequency content of the P and R-waves in the mouse ECG waveform may cause a P wave to be misinterpreted as an R-wave in slower heart rates, causing an algorithm designed for human optimization to fail for processing mouse ECG signals. A trigger hold-off parameter for a human ECG process is set much longer than the average cycle or R-R interval of a mouse's heart rate, causing R-waves for a mouse's ECG waveform to be missed. Different cycle processes are provided for different sources (e.g. human versus mouse) or conditions (e.g., normal versus abnormal) for optimal processing and greater usefulness for the different types of waveforms. While a mouse versus human examples is given above, a normal versus abnormal, human versus another animal or other types of waveforms may be individually or separately optimized using different processing, such as different parameter settings or algorithms.

In one embodiment, a given process is operable with different sources or conditions, such as both human and mouse waveforms. For example, the process implements a relative local-to-peak analysis to identify R-wave triggers. However, the processes may be optimized for other waveform processes, such as for generating a display of the waveform.

The processor 22 is a single or multiple processors. The processor 22 is an application-specific integrated circuit, general processor, control processor, digital signal processor, field programmable gate array, digital circuit, analog circuit, circuit boards combinations thereof or other now known or later developed device for processing analog or digital signals. The processor 22 is operable to implement at least one of different physiological waveform processes on the signals received at the input 18. For example, the processor 22 provides two different paths for implementing two different types of processes. The processor 22 provides one path for detecting triggers from the waveform, and a separate path for generating a display of the waveform. The detected waveform signals are routed through a user interface system controller or software for display of the waveform on a display 32. The other path is responsive to the user interface to detect events associated with the physiological cycle, such as an R-wave of the ECG. The processor 22 may archive detected events, detected waveforms or other physiological data in the memory 20. Any of various communications between different portions of the processor 22 may be used, such as serial or parallel bus communication. In one embodiment, the path for generating a waveform for display includes a band pass filter, a decimator and a gain amplifier. Additional, different or fewer components may be provided. In one embodiment, the path for detecting physiological cycle events includes one or more filters, such as a one-pole high pass butterworth filter followed by a one-pole high pass butterworth filter with a different frequency followed by a three-pole low pass butterworth filter. A processor for detecting information from the filtered signals may also be provided. Additional, different or fewer components may be provided, such as using different filters. While two separate paths are used, a common path may provide for detection of the waveform for display and detection of each cycle events based on the waveform in a single path.

For a given type of processing, the processor 22 is operable to implement one or more different physiological waveform processes on the signals. For example, the processor 22 is operable to apply a different algorithm or different settings of parameters for applying a same type of process to different physiological waveforms (e.g. mouse versus human). The processor 22 is operable to implement different physiological waveform processes, such as by using different parameters or settings in one of the paths discussed above. For example, cutoff frequencies of the filters in the detection path are 1.5 Hertz for the first filter to eliminate DC components, 35 Hertz in the low pass filter to reduce noise and 23 Hertz in the middle filter to reduce the high amplitude T and P waves, avoiding false positives for R-wave trigger events. A sudden relative change from a low value to a peak indicates an R-wave in the output signal. A peak amplitude threshold or other techniques may alternatively be used to detect the R-wave or other portion of the waveform. A hold-off time period used for human ECG signals is based on the human heart rate. By avoiding detecting an R-wave or other event for a particular period of time, one or two of the events of a higher heart rate mouse may be bypassed without detection. Similarly, other time periods may be optimized to a normal human ECG, such as a time period associated with errors. A “no trigger found” message may be generated after a sufficient period of attempting to and not locating a trigger. A period may be different for different types of waveforms.

For a mouse ECG, the 35 Hertz low pass filter may suppress the QRS peak and the 23 Hertz high pass filter may fail to suppress the P and T waves sufficiently. The P and R-wave characteristics may be so similar that single non-adaptive filters may fail to differentiate between P and R-waves. The parameters, such as the filter cutoff frequencies, heart rate limits, filter bandwidth, trigger hold-off times, trigger threshold or other settings as well as the algorithm applied, such as the number of filters used, is optimized for a mouse ECG in the example above. By altering the software associated with the event detection path, the user interface and any intercommunications, the processor 22 implements a physiological waveform process appropriate for a particular source or condition in physiological waveform signals. In one embodiment, four alternative cycle detection processes are provided for rodent or mouse ECG signals in addition to one for a human. Each of the four processes is associated with a different set of filters, filter parameters, trigger hold-off times and/or trigger time-out times. A same or a different path is provided for implementing the different processes.

In one embodiment, the same filters and filter parameters are implemented for small animal ECG event detection, but with different hold-offs or other time-based triggers. For example, the trigger hold-off time or sleep mode after a particular event has been detected is reduced, allowing a lesser R-R interval for example. For human detection, the hold-off trigger interval is set at about 180 milliseconds, but the time is adjusted to 60 milliseconds for a small animal processing. The “no trigger found” time period for sending an error message may be 2,050 milliseconds for normal human ECG processing and 1,000 milliseconds for small animal ECG processing. Other time periods may be used.

Another small animal filter removes the 23 Hertz high pass filter component but maintains the other filtering. The timers are also adjusted as discussed above. In yet another optimization, higher cut-off frequencies are used in the low pass filter, such as 135 Hertz or 345 Hertz. The high pass filter components are the same as for the human processes. Higher cut-off frequencies for the low pass filter allow for higher frequency content in the QRS waveform decreasing false positives. Different parameters, changes, settings, algorithms or other features may be used for the same source and condition or between different sources and conditions of waveforms.

Using the selected physiological cycle process, the processor 22 detects an event, such as an ECG R-wave, associated with a particular source, such as a small animal or human. The processor 22 may generate an output for use in triggering of the imaging system 14.

The output 26 is a mechanical connector or electrical communications device. The signals on the output 26 may also be used by the processor 22 to select a different physiological waveform process as a function of the signals. For example, a characteristic of the input signals is determined, and a desired physiological waveform process is selected in response to the characteristic. In the mouse versus human example above, the heart rate may indicate whether the source is a mouse or a human. Once the rate is determined, the processor 22 is operable to select the appropriate physiological cycle process based on the characteristic.

The user interface 24 is a keyboard, buttons, knobs, dials, sliders, mouse, trackball, touchpad, touch screen or other now known or later developed user input devices. The processor 22 is responsive directly or through a user interface control processor to input from the user input 24. As an alternative to the processor 22 selecting different physiological waveform processes without user input, the processor 22 selects one of the different physiological waveform processes in response to user input. For example, a soft key (e.g. a button adjacent to a display) for selecting or switching between different processes is provided. By depressing the button, the processor 22 automatically switches between the different processes by loading the process out of the memory 20. A list of selectable processes, a dial for selecting between multiple processes or other user inputs may be used for selecting one of the different physiological waveform processes. For example, the user selects an imaging application associated with imaging of a particular source and/or condition, such as a human with an abnormal ECG or a small animal. In response to the selection of the application, the processor 22 implements an associated physiological waveform process for that source. For example, the selection of a human ECG is associated with a minimal and a maximum allowed heart rate of 30 and 300 beats per minute, but the selection of imaging of a small animal results in a minimum and maximum heart rates of 150 and 999 beats per minute, respectively.

The imaging system 14 includes a beamformer 28, an image processor 30 and a display 32. Additional, different or fewer components may be provided. The beamformer 28 is operable to ultrasonically scan a region. In response to the received echo signals, the image processor 30 detects and generates an image of the scanned region. For example, Doppler detection and scan conversion are provided. The generated image is then provided on the display 32. In one embodiment, Doppler signals are provided to the input 18 for use in generating a waveform and detecting events within a cycle. Where a physiological waveform is output by the output 26, the waveform may be displayed on the display 32 with the image or with a sequence of images. Detected events associated with a physiological cycle may also be indicated on the waveform or separately on the display 32. In one embodiment, the imaging system 14 responds to triggers of the output 26. The beamformer 28 is responsive to trigger signals to cause a transmission of ultrasonic energies. A scan is performed in response to a trigger and no scanning is provided in between the triggers. Such triggering may be used in contrast agent studies where acoustic energy may destroy contrast agents. The triggering minimizes destruction of the contrast agents and allows reperfusion. By providing triggering that is more accurate through a particular physiological waveform process specific to the source of signals or condition, the triggering may be more optimal.

FIG. 2 shows one embodiment of a method for processing signals representing a physiological cycle with an ultrasound system. Different, additional or fewer acts may be provided in the same or different order than shown in FIG. 2. For example, the acts 56 and 60 may be skipped. As another example, one of the acts 46 and 48 is skipped as part of the act 44, or one of the acts 52 and 54 are skipped as part of the act 50. The method shown in FIG. 2 is implemented with the system 10 of FIG. 1 or a different system.

In act 40, signals representative of at least a portion of a physiological cycle are received. For example, ECG or breathing cycle signals are received. ECG signals are received from ECG sensor, such as electrodes in an associated processor for converting the ECG electrode signals into differential signals. Breathing cycle signals may be used for respiratory gating. For example, three-dimensional imaging is performed at a same portion of a respiratory cycle to avoid misregistration of image information. Transthoracic impedance information, acoustics, pulse measurements or nasal thermistor information may be used as breathing cycle signals. Other ECG or breathing cycle signals may be used. Other physiological cycles may be tracked or detected, such as brain waves.

In act 42, at least two different parameters associated with respective two different physiological cycle characteristics are provided. Different physiological cycle characteristics correspond to different sources or conditions of the physiological cycle. For example, different sets of parameters associated with normal ECG waveforms and abnormal ECG waveforms are provided. As another example, two sets of different parameters are associated with a human physiological cycle and a non-human physiological cycle. Two different sets for human physiological cycles may be provided. The sets may have some of the same parameters or settings, but at least one parameter or setting is different. The different parameters are associated with different processes for the same type of physiological cycle. For example, animal types include small animals, such as rats, mice or rabbits; large animals such as horses, dolphins or whales; monkeys; babies; fetuses; and different human pathological conditions such as tachycardia or arrhythmia. There is a different characteristic for the physiological waveforms of each of these animal types. The characteristics of the waveform and the dynamics of the heart are used to tune the various parameters or associated processes to optimize performance.

Any resolution of optimization may be provided, such as providing for different parameters for each of the above listed sources, conditions or associated physiological cycle characteristics. Alternatively, as few as two different sets of parameters are provided, such as one for human and large animals and another for small animals. While ECG waveforms and associated differences between sources are discussed above, breathing cycle or other physiological waveforms may differ as a function of any of various characteristics of the source. Different parameter sets may be provided for different ones of the variations for the same type of physiological cycle.

In act 44, one of the different parameters is selected. For example, a selection of one parameter over another parameter is performed in response to the source of the physiological cycle waveform, such as a human with an abnormal heart condition versus a human with a normal heart condition. As another example, a parameter associated with a mouse physiological cycle is selected over a parameter associated with a same type of human physiological cycle. The selection is performed automatically by a processor in act 46 or in response to user input in act 48.

In act 46, the processor or system automatically detects the source of the physiological cycle signals. To detect the source, a characteristic of the signals is determined. For example, ECG signals are examined for one or more different characteristics determinative of the source. In one embodiment, the triggering or a waveform detection process is performed with parameters having inclusive settings, such as parameters allowing ranges of heart rates inclusive of multiple different sources. By then determining the heart rate, the source is identified. Other characteristics than heart rate may be used, such as intervals between any two portions of an ECG waveform, an amplitude of a maximum pulse within a waveform. In one embodiment, a small animal respiration is detected as opposed to other sources of respiration cycle signals by a short burst of electromyographic noise. When rodents breathe, a muscle contraction causes short bursts of electromyographic noise in the ECG waveform. By performing filtering and spectral analysis of the ECG waveform designed to identify the electromyographic noise, the source can be determined as a small animal or rodent. The spacing or location of the noise bursts in time may be used to estimate the breathing rate or to trigger for cardiac image acquisition. The electromyographic noise can also be used for humans.

By detecting one of two different physiological cycle characteristics, a processor can select the appropriate source. For example, characteristics associated with a normal human physiological cycle, an abnormal human physiological cycle, a first type of non-human physiological cycle, or a second type of non-human physiological cycle are provided. The appropriate parameters for more exclusive settings or processes may then be selected. In one embodiment, the selection is performed by comparing the receive signals to different characteristics or characteristic ranges for the different sources. As an alternative to using inclusive settings, exclusive settings are used to rule out different sources or identify a particular source.

The processing of the signals is then set as a function of the determined characteristic. For example, a parameter associated with the detected physiological cycle characteristic is selected. The processing of the signal is then altered to be less inclusive or more exclusive for processing signals from the determined source or condition. Any of the processing performed, such as detection of events or generations of waveforms, may be altered. In the embodiment above, the determination of the source and the processing performed in response to the determined characteristic is the same. In alternative embodiments, one process is used for detecting the characteristic and a separate process is then altered based on the detected characteristic.

In act 48, the selection of the parameter in act 44 is performed in response to input by a user. For example, a single touch of a button or a single user activation causes the selection of different parameter or parameters. By activating a button, the event detection and/or waveform processes are altered as appropriate for a different source, such as changing from a human process to a mouse process or vice versa. As another example, processing switches using parameters for normal and abnormal conditions. The single user activation or a plurality of user activations may be used to select between two or more different parameters or parameter sets. For example, a dropdown menu is provided for selecting between the different parameters and/or different sources or conditions. For example, four different filter or parameter sets are provided for a same source or for different conditions from a same source. As another example, two or more different sources are listed. A message associated with the available options or selected parameters may be displayed, such as a maximum heart rate. In one embodiment, a default setting is provided, such as a normal human processing and an associated parameter set.

In act 50, the physiological cycle signals are processed. The processing is performed as a function of the selected parameters. In one embodiment, the processing is the detection of cycle events in act 52, such as for triggering. Processing may include filtering of signals. For example, the parameter selected is one of different filter parameters, such as filter bandwidths or cutoff frequencies. Other parameters may include different combinations of filters. Yet other parameters may include selecting between different heart rate limits, different trigger hold-off times, different trigger thresholds or combinations of any of the various parameter types discussed herein. The selected ones of the different parameters are then applied.

FIG. 5 shows one embodiment of an implementation of a trigger hold-off time. Trigger hold-off time is used for detecting cycle events, such as an R-wave. Different trigger hold-offs may be used for different sources or physiological cycle processes. In act 70, it is determined whether the hold-off time has expired. If the hold-off period is not yet greater than zero after the last detected event, the process maintains itself at act 70. If the hold-off period has now expired, the process sets a peak value to be 0.68 of the peak in act 72. In act 74, the current input value is compared to 0.91 of the current peak setting. If the absolute value of the input is greater than the peak threshold times 0.91, a trigger is activated in act 76. If not, the process continues to act 72 through act 74 until a peak with sufficient amplitude is identified. In one embodiment, the peak value is continually decreased for each repetition, but may be maintained at a same value in other embodiments. Once a trigger is identified in act 76, the hold-off period begins again and repeats to act 70. Triggers are not searched for until the hold-off period has expired. The hold-off period avoids detecting peaks other than R-wave signals. The processing of signals is performed in response to selected one of different trigger hold-off times. A 60 millisecond trigger hold-off time may be used for small animals, but a greater 180 milliseconds is used for humans given the different likely intervals between R-waves.

In an alternative or additional embodiment, the processing of the signals as a function of the selected parameters is processing for generating a waveform representing the physiological cycle in act 54. Any of various parameters used, such as the band pass filtering parameters, gain parameters, decimation parameters or other settings as well as differences in algorithms may be selected for different types of animals or conditions. The generation of the waveform process is then altered as a function of the selected parameter set.

In act 56, transmit power calculations are altered as a function of the selection of different parameters or other determination of the source or condition associated with the physiological cycle signals. Beamformer power management for triggered imaging modes may take into account maximum heart rate. Since the maximum heart rate may vary between different types of animals or different sources, the power calculations associated with the heart rate limit may be altered or maintained but with a new heart rate limit corresponding to the identified source. In one embodiment, power management is not altered in non-triggered imaging, but may be altered in triggered imaging. Power management limits the power transmitted by the beamformer 28. If low power contrast agent imaging is used, the power management may not be altered. However, for higher power contrast agent imaging, a reduction in the transmit power may be used given the increase in heart rate to allow contrast agent sufficient time to re-perfuse a scan region. Higher trigger rates result in higher frame rates, and thus more power delivered to the body. If the maximum allowed trigger rate is modified, a decrease in the transmitted power may be used in order not to harm tissue or exceed regulated limits. For example, with heart rates between 300 and 599 beats per minute, a 3 dB reduction may be provided. For heart rates from 600-999 beats per minute, a 6 dB down reduction in power may be used. Other heart rate ranges and/or associated amounts of power reduction may be used. The transmit power is set in triggered imaging based on the maximum heart rate anticipated. The maximum heart rate anticipated is indicated by the selection of the different parameters associated with the different sources or conditions. Power adjustments associated with other physiological cycles may be provided.

In act 60, other imaging is adjusted as a function of the selection of different parameters. For example, the source of the physiological cycle signals is determined automatically or by user input. In response to the determination, ultrasound imaging parameters are selected as well as physiological cycle processing parameters. In one embodiment, a user selects a small animal imaging application. Both the physiological cycle processing as well system presets for imaging associated with small animals are then selected and used. The frame rate may be increased and a system center frequency for transmit and reception of acoustic energy increased for small animals. As another example, physiological cycle signals are examined to determine the source or a characteristic of the signals. In response to the determination, parameters are selected, such as selecting a small animal physiological processes and imaging settings.

Selection may also be used for annotation of archived records or displayed information. For example, the identified or selected source or condition of the source is displayed. Where the user believes the system has performed erroneously, the source or condition of the source may be altered through user input, more likely optimizing the physiological cycle processing and ultrasound image processing.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for processing signals representing a physiological cycle with an ultrasound system, the method comprising:
(a) receiving signals representative of at least a portion of a physiological cycle;
(b) providing at least two different parameters associated with a respective two different physiological cycle characteristics;
(c) selecting one of the at least two different parameters; and
(d) processing the signals as a function of the selected one of the at least two different parameters.

2. The method of claim 1 wherein (b) comprises providing the at least two different parameters associated with normal physiological cycle and an abnormal physiological cycle.

3. The method of claim 1 wherein (b) comprises providing the at least two different parameters associated with a human physiological cycle and a non-human physiological cycle.

4. The method of claim 1 wherein (a) comprises receiving one of ECG or breathing cycle signals.

5. The method of claim 1 wherein (c) comprises:
(c1) detecting one of the two different physiological cycle characteristics with a processor; and
(c2) selecting the associated parameter based on the detected one of the physiological cycle characteristics.

6. The method of claim 1 wherein (c) comprises selecting in response to input by a user.

7. The method of claim 6 wherein (c) comprises selecting in response to a single user activation.

8. The method of claim 1 wherein (d) comprises detecting a cycle event.

9. The method of claim 1 wherein (d) comprises generating a waveform representing the physiological cycle.

10. The method of claim 1 wherein the at least two different parameters are at least two different filter parameters and (d) comprises filtering the signals.

11. The method of claim 1 wherein the at least two different parameters are at least two different combinations of filters and (d) comprises filtering the signals.

12. The method of claim 1 wherein the at least two different parameters are at least two different settings of: heart rate limits, filter bandwidths, cutoff frequencies, trigger hold-off times, trigger thresholds or combinations thereof, and (d) comprises applying one of the at least two different settings.

13. The method of claim 1 further comprising:
(e) altering a transmit power as a function of the selection of (c).

14. The method of claim 1 further comprising:
(e) adjusting ultrasound imaging as a function of the selection of (c).

15. A system for optimizing an ultrasound system for a specific physiological waveform, the system comprising:
an input operable to receive signals representative of at least a portion of a physiological cycle;
a memory operable to store different physiological waveform processes for different physiological waveforms of a same type; and
a processor operable to implement at least one of the different physiological waveform processes on the signals.

16. The system of claim 15 wherein the different physiological waveform processes are different settings for a same algorithm.

17. The system of claim 15 wherein the different physiological waveform processes are different settings selected from the group of: filter parameters, combinations of filters, heart rate limits, filter bandwidths, cutoff frequencies, trigger hold-off times, trigger thresholds or combinations thereof.

18. The system of claim 15 wherein the different physiological waveforms comprise waveforms selected from the group of: a normal physiological cycle, an abnormal physiological cycle, a human physiological cycle and a non-human physiological cycle.

19. The system of claim 15 wherein the input comprises an ECG input.

20. The system of claim 15 wherein the processor is operable to select the one of the different physiological waveform processes as a function of the signals.

21. The system of claim 15 further comprising:
a user input, wherein the processor is responsive to input from the user input to select the one of the different physiological waveform processes.

22. A method for processing signals representing a physiological cycle with an ultrasound system, the method comprising:
(a) receiving signals representative of at least a portion of a physiological cycle;
(b) determining a characteristic of the signals;
(c) processing the signals; and
(d) setting the processing of (c) as a function of the characteristic.

23. The method of claim 22 wherein (a) comprises receiving ECG signals, wherein (b) comprises determining heart rate, wherein (c) comprises one of triggering and generating a waveform, and wherein (d) comprises altering a setting selected from the group of: filter parameter, combination of filters, heart rate limit, filter bandwidth, cutoff frequency, trigger hold-off time, trigger threshold or combinations thereof.

24. The method of claim 22 wherein (b) comprises identifying the signals as waveform types of: a normal-human physiological cycle, an abnormal-human physiological cycle, a first type of non-human physiological cycle, or a second type of non-human physiological cycle, the determination being based on comparisons for at least two of the waveform types.

25. The method of claim 22 wherein (b) comprises processing the signals with inclusive settings and wherein (d) comprises altering the processing of (c) to have less inclusive settings, the processing of (b) and (d) being a same type of processing.

26. The method of claim 22 wherein (b) comprises detecting a electromyographic noise.

27. A method for processing signals representing a physiological cycle with an ultrasound system, the method comprising:
(a) receiving signals representative of at least a portion of a physiological cycle;
(b) providing at first and second different parameters associated with a respective two different physiological cycle characteristics;
(c) switching between processing the signals with the first and the second different parameters in response to user input.

28. The method of claim 27 wherein (a) comprises receiving ECG signals, wherein (b) comprises providing the first parameter for a normal ECG waveform and providing the second parameter for an abnormal ECG waveform, and wherein (c) comprises switching between processing the signals as normal and abnormal.

29. The method of claim 27 wherein (b) comprises providing the first parameter for a human waveform and providing the second parameter for a non-human waveform.

30. The method of claim 27 wherein (c) comprises switching in response to a single user activation.
